(19) United States
(12) Patent Application Publication
(10) Pub. No.: US 2024/0025999 A1
(43) Pub. Date: Jan. 25, 2024

(54) T CELLS AGAINST HUMAN PAPILLOMAVIRUS

(71) Applicant: BioVentures, LLC, Little Rock, AR (US)

(72) Inventor: Mayumi Nakagawa, Little Rock, AR (US)

(21) Appl. No.: 18/144,142

(22) Filed: May 5, 2023

Related U.S. Application Data
(63) Continuation of application No. PCT/US21/58284, filed on Nov. 5, 2021.
(60) Provisional application No. 63/110,813, filed on Nov. 6, 2020.

(51) Int. Cl. C07K 16/28; C12N 5/0783; C07K 16/08; A61P 35/00; A61K 35/17

(52) U.S. Cl. CPC C07K 16/2809; C12N 5/0636; C07K 16/084; A61P 35/00; A61K 35/17; C12N 2510/00; C07K 2317/622; C07K 2317/565; C07K 2317/31

(57) ABSTRACT

Recombinant T cell clonotypes are provided that express T cell receptor alpha and T cell receptor beta polypeptides with specificity for human papillomavirus (HPV) type 16 E6 protein and that amplify in response to a therapeutic vaccine and traffic to ovarian lesional tissue in a patient whose HPV lesions regressed in response to the vaccine. Recombinant T cells expressing appropriate TCR alpha and beta complimentarity determining sequences for HPV 16 E6 binding and treating HPV-cased cancers are provided. Bifunctional proteins having TCR alpha and beta segments that bind to HPV 16 E6 residues 91-115 and a single chain Fv anti-CD3 antibody domain are provided. These bifunctional proteins can direct T cells to HPV-infected cells.

### BACKGROUND

Human papillomavirus (HPV) is best known as the causative agent of cervical cancer, but it can also cause cancers at other mucosal sites including the anus, oropharynx, penis, vagina, and vulva. It is estimated that HPV is responsible for 42,700 cancers in the US each year (1), including more than 90% of anal and cervical cancers and about 70% of oropharyngeal, vaginal, and vulvar cancers (1). Incidences of HPV-associated anal and oropharyngeal cancers have increased notably, and, although incidence of cervical cancer has stabilized after significant decreases over the past several decades (2), this remains the fourth most common cancer among women globally (3). The available prophylactic vaccines are effective for preventing HPV infections, but they cannot eliminate established infections; therapeutic vaccines could fill this need, but none are currently available (4). Such vaccines would benefit young women (narrowly, those <24 years old) and, broadly, any woman who plans to become pregnant (5) because increased incidence of preterm delivery (from 4.4% to 8.9%) is associated with surgical treatments (e.g., loop electrical excision procedure [LEEP]) for high-grade squamous intraepithelial lesion (HSIL) (5,6).

We have previously evaluated the safety of an HPV therapeutic vaccine (PepCan) in a single center, single arm, dose-escalation Phase I clinical treating women with biopsy-proven HSILs (NCT01653249) (7,8). PepCan consists of four current good manufacturing practice (cGMP)-grade peptides covering the human papillomavirus type 16 (HPV 16) E6 protein (amino acids 1-45, 46-80, 81-115, and 116-158) and Candida albicans skin test reagent (Candin®, Nielsen Biosciences, San Diego, CA). PepCan was shown to be safe, resulted in histological regression rate of 45% which is roughly double that of a historical placebo (22%) (9). In addition, circulating T-helper type I (Th1) cells in periphery (p=0.0004) was increased, and the HPV 16 viral load was significantly decreased (p=0.008) (7).

New therapies for human papilloma virus-caused diseases and particularly HPV-caused cancers are needed.

### SUMMARY

The inventor has shown that PepCan, a therapeutic vaccine comprising four peptides derived from human papillomavirus type 16 (HPV 16) E6 protein (amino acids 1-45, 46-80, 81-115, and 116-158) combined with Candida albicans skin test reagent, caused resolution of cervical high-grade squamous intraepithelial lesions in a significant number of patients. HPV-specific T cells are characterized from one patient who experienced complete resolution of cervical high-grade squamous intraepithelial lesions after dosing with the PepCan vaccine. In particular, T cell clonotypes that were amplified after the vaccine and localized to cervical tissue and expressed interferon-gamma and bound to HPV 16 E6 91-115 (which was the most recognized peptide) were identified. The T cell receptor (TCR) alpha and beta chains and the variable regions of the TCRA and TCRB chains in these clonotypes were determined.

The invention provides isolated recombinant T cells expressing certain TCR alpha and/or TCR beta sequences that bind to HPV 16 E6 protein, particularly in the residues 91-115 of E6.

Other embodiments provided are methods of identifying T cell clonotypes that respond to a vaccine.

Another embodiment provides a bifunctional protein comprising a CD3-binding domain and an HPV E6 91-115-binding domain.

One embodiment provides a plurality of isolated recombinant human T cells enriched for recombinant human T cells comprising a recombinant T cell receptor beta (TCRB) gene encoding CDR3 sequence CASSPTSGGLTWDEQYF (SEQ ID NO:1) or CASSHNSGREGNEQFF (SEQ ID NO:2) or CASSFPGENEQFF (SEQ ID NO:3) or CASSWEAGQETQYF (SEQ ID NO:4).

One embodiment provides a method of identifying T cell clonotypes that respond to a vaccine, the method comprising: (a) isolating T cells from a human who has been vaccinated with an immunogen comprising a polypeptide or plurality of peptides; (b) stimulating the T cells with one or more peptides that collectively constitute less than all of the sequence of the polypeptide or less than all of the plurality of peptides; (c) identifying one or more T cell clones that are stimulated by the one or more peptides by a process comprising an interferon gamma (IFNG) enzyme-linked immunospot (ELISPOT) assay; and (d) determining a nucleotide sequence of T cell receptor (TCR) alpha or T cell receptor beta gene or a segment of TCR alpha or TCR beta of the one or more clones to identify a clonotype having a particular TCR alpha or beta gene or a variable region segment of a TCR alpha or beta gene.

Another embodiment provides a population of recombinant T cells prepared by a process comprising: (1) identifying a human T cell clonotype that specifically binds an identified portion of HPV E6 or E7 polypeptides by a process comprising: (a) isolating T cells from a human who has been vaccinated with an immunogen comprising an HPV E6 or E7 polypeptide or a plurality of peptides from HPV E6 or E7; (b) stimulating the T cells with one or more peptides that collectively constitute less than all of the sequence of the polypeptide or less than all of the plurality of peptides; (c) identifying one or more T cell clones that are stimulated by the one or more peptides; and (d) determining a nucleotide sequence of T cell receptor (TCR) alpha or TCR beta gene or a segment of TCR alpha or TCR beta of the one or more clones to identify a clonotype having a particular TCR alpha or beta gene or a variable region segment of TCR alpha or beta gene; (2) producing a recombinant vector encoding TCR alpha or beta or both or a segment thereof corresponding to the clonotype; and (3) transfecting human T cells ex vivo with the vector to generate recombinant T cells that specifically respond to HPV E6 or E7 or a portion of HPV E6 or E7.

Other embodiments provide methods of treating a human for a cancer caused by HPV comprising administering T cells or bifunctional proteins of the invention.

### BRIEF DESCRIPTION OF THE DRAWINGS

FIG. 1. Design and sequence of a bispecific protein.

FIG. 2. The Phase I clinical trial design and routine immune monitoring assays. Clinical trial design of the Phase I study. Vaccination (PepCan) visits were scheduled 3 weeks apart for patients who had biopsy-confirmed cervical high-grade squamous intraepithelial lesions (HSILs, i.e. CIN grade 2 or 3). Blood draws were performed in the pre-vaccine phase, post-2-vaccines phase, and post-4-vaccines phase. Cervical local samples for LBC samples and FFPE samples were collected in the pre-vaccine phase and post-4-vaccines phase. FFPE samples were taken from a cervical biopsy in the pre-vaccine phase and loop electrical excision procedure (LEEP) biopsy in the post-4-vaccines phase.

FIG. 3. Immunogenic HPV16 E6 and E7 regions were determined for each vaccine phase using ELISPOT assay. In pre-vaccine phase, positive responses (i.e., at least twice the media control) were detected in the E6 16-40, E6 31-55, and E6 106-130 regions. Positive responses were seen in the E6 1-25, E6 106-130, E6 136-158, and E7 1-regions in the post-2 vaccination sample, and in the E6 31-55, E6 91-115, and E7 46-regions in the post-4 vaccination sample. The increase in the response to the HPV16 E6 91-115 regions was statistically significant (paired t-test, p=0.023). Phytohemagglutinin was used as a positive control (not shown). The bars represent mean spot forming units per 1×10⁶ CD3 T cells, and the error bars represent standard error of means.

FIG. 4. The Th1 level expressed as the percentage of CD4 T cells increased after 4 vaccinations, but the Treg and Th2 levels were minimally changed.

FIG. 5. T cell structures of PBMC stimulated CD3, LBC, and FFPE samples described with multiplexed PCR-based TCR sequencing using genomic DNA. A. The T cell structures of the 4 sample types (PBMC, stimulated CD3, LBC, and FFFPE) are shown as the total number of T cells defined by nucleotides, productive clonality, fraction of T cells, number of clonotypes defined by nucleotides, maximum productive frequency, and the quantity of DNA (ng) required per cell.

FIG. 6. Tracking of the top 15 clonotypes defined by nucleotides are shown in productive frequency. The cumulative frequency of the top 15 clonotypes is shown. The top 15 highest frequency clonotypes were significantly decreased after 2 vaccinations (Wilcoxon matched-pairs singed-ranks sum test, p<0.0001), but significantly increased after 4 vaccinations (p<0.0001) in PBMC samples calculated using the numbers of T cells. For stimulated CD3 T cell samples, significant increases were seen between pre-vaccination and post-4 vaccination (p=0.034) and between post-2 and post-4 vaccination samples (p=0.0034). Significant increase was seen in the LBC samples (p<0.0001) but not in FFPE samples.

FIG. 7. Venn diagrams of clonotypes defined by nucleotides in PBMC, LBC, and FFPE samples in pre-vaccination visit and post-4 vaccination visits. Most clonotypes appear only in one sample type, but there are 17 TCRs present at the pre-vaccination visit and 72 TCRs at the post-4 vaccination visit.

FIG. 8. Tracking HPV 16 E6 91-115 specific T cells in PBMC, LBC, and FFPE. The frequency of the four most common clonotypes from Table 3 pre-vaccination and after 2 and 4 vaccinations is shown.

FIG. 9. Complete TCR alpha and TCR beta encoded proteins of clonotypes 1-4 of Table 3.

### DETAILED DESCRIPTION

The inventor has identified T cell clonotypes that are amplified by an effective HPV treatment vaccine and traffic to diseased cervical tissue and recognize an epitope of the HPV E6 protein used in the vaccine. T cells expressing these clonotypes are expected to be effective to treat HPV disease, including HPV-caused cancer.

One embodiment provides isolated recombinant T cells expressing certain TCR alpha and/or TCR beta sequences that bind to HPV 16 E6 protein, particularly in the residues 91-115 of E6.

One embodiment provides a plurality of isolated recombinant human T cells enriched for recombinant human T cells comprising a recombinant T cell receptor beta (TCRB) gene encoding CDR3 sequence CASSPTSGGLTWDEQYF (SEQ ID NO:1) or CASSHNSGREGNEQFF (SEQ ID NO:2) or CASSFPGENEQFF (SEQ ID NO:3) or CASSWEAGQETQYF (SEQ ID NO:4). These TCRB CDR3 sequences were found in clonotypes 1-4 described in Example 1. The TCRB CDR3 sequences are generally believed to confer the epitope recognition specificity, and thus could be switched to other full-length TCRB proteins and paired with different TCRA proteins and still confer recognition of the same epitope.

Another embodiment provides a plurality of isolated recombinant human T cells enriched for recombinant human T cells comprising a recombinant T cell receptor alpha (TCRA) gene encoding CDR3 sequence CAPRVTGGGNKLTF (SEQ ID NO:5), CAVRDQRDDKIIF (SEQ ID NO:6), CAVRAPSGSARQLTF (SEQ ID NO:7), CALTLSGSARQLTF (SEQ ID NO:8), or CAASAPGRTDKLIF (SEQ ID NO:9). Those are the TCRA CDR3 sequences of clonotypes 1-4 of Example 1.

Another embodiment provides a plurality of isolated recombinant human T cells enriched for recombinant human T cells comprising: (a) a recombinant T cell receptor alpha (TCRA) gene encoding CDR3 sequence SEQ ID NO:5 and a T cell receptor beta (TCRB) gene encoding CDR3 sequence SEQ ID NO:2; (b) a recombinant TCRA gene encoding CDR3 sequence SEQ ID NO:6 or 7 and a recombinant TCRB gene encoding CDR3 sequence SEQ ID NO:1; (c) a recombinant TCRA gene encoding CDR3 sequence SEQ ID NO:8 and a recombinant TCRB gene encoding CDR3 sequence SEQ ID NO:3; or (d) a recombinant TCRA gene encoding CDR3 sequence SEQ ID NO:9 and a recombinant TCRB gene encoding CDR3 sequence SEQ ID NO:4.

In specific embodiments with the full-length TCRA and TCRB genes found together in the four clonotypes of Example 1, the isolated recombinant human T cells comprise: (a) a recombinant T cell receptor alpha (TCRA) gene encoding SEQ ID NO:10 and a TCRB gene encoding SEQ ID NO:11; (b) a TCRA gene encoding SEQ ID NO:12 or SEQ ID NO:13 and a TCRB gene encoding SEQ ID NO:14; (c) a TCRA gene encoding SEQ ID NO:15 and a TCRB gene encoding SEQ ID NO:16; or (d) a TCRA gene encoding SEQ ID NO:17 and a TCRB gene encoding SEQ ID NO:18.

Another embodiment provides a method of identifying T cell clonotypes that respond to a vaccine, the method comprising:
(a) isolating T cells from a human who has been vaccinated with an immunogen comprising a polypeptide or plurality of peptides;
(b) stimulating the T cells with one or more peptides that collectively constitute less than all of the sequence of the polypeptide or less than all of the plurality of peptides;
(c) identifying one or more T cell clones that are stimulated by the one or more peptides by a process comprising an interferon gamma (IFNG) enzyme-linked immunospot (ELISPOT) assay; and
(d) determining a nucleotide sequence of T cell receptor (TCR) alpha or T cell receptor beta gene or a segment of TCR alpha or TCR beta of the one or more clones to identify a clonotype having a particular TCR alpha or beta gene or a variable region segment of a TCR alpha or beta gene.

In a more specific embodiment of this method, the immunogen comprises human papilloma virus (HPV) E6 protein or a portion of the HPV E6 protein.

In another specific embodiment of the method, step (d) comprises determining the nucleotide sequence of CDR3 variable region segment of TCR beta.

In other specific embodiments of the method, steps (a)-(d) are conducted on T cells collected from a tumor sample or HPV-infected diseased tissue and from T cells collected distal from a tumor or HPV-infected disease tissue, and the method further comprises comparing frequency of the identified clonotypes between the tumor or diseased tissue and the distal tissue samples to identify clonotypes more common in the tumor or diseased tissue samples.

In other specific embodiments of the method, the method further comprises collecting T cells before the human is vaccinated with the immunogen and conducting steps (b)-(d) and later conducting steps (a)-(d) after the human has been vaccinated with the immunogen, and the method further comprises comparing frequency of the identified clonotypes before and after the human is vaccinated with the immunogen to identify clonotypes more common after vaccination than before.

Another embodiment provides a population of recombinant T cells prepared by a process comprising:
(1) identifying a human T cell clonotype that specifically binds an identified portion of HPV E6 or E7 polypeptides by a process comprising:
   (a) isolating T cells from a human who has been vaccinated with an immunogen comprising an HPV E6 or E7 polypeptide or a plurality of peptides from HPV E6 or E7;
   (b) stimulating the T cells with one or more peptides that collectively constitute less than all of the sequence of the polypeptide or less than all of the plurality of peptides;
   (c) identifying one or more T cell clones that are stimulated by the one or more peptides; and
   (d) determining a nucleotide sequence of T cell receptor (TCR) alpha or TCR beta gene or a segment of TCR alpha or TCR beta of the one or more clones to identify a clonotype having a particular TCR alpha or beta gene or a variable region segment of TCR alpha or beta gene;
(2) producing a recombinant vector encoding TCR alpha or beta or both or a segment thereof corresponding to the clonotype; and
(3) transfecting human T cells ex vivo with the vector to generate recombinant T cells that specifically respond to HPV E6 or E7 or a portion of HPV E6 or E7. The term “transfecting” as used herein includes any means of introducing DNA into a cell, including by chemical or physical means or by transduction or infection of a target cell with a virus or phage.

In specific embodiments of the recombinant T cells, step (c) comprises a process comprising an interferon gamma (IFNG) enzyme-linked immunospot (ELISPOT) assay.

In specific embodiments of the recombinant T cells, the preparation process further comprises amplifying the recombinant T cells ex vivo.

Another embodiment of the invention provides a method of treating a human for a cancer or other disease caused by human papilloma virus (HPV) comprising: administering the T cells of any of the embodiments described herein to a human afflicted with a cancer caused by HPV or with another disease caused by HPV (e.g., precancerous lesions).

In one embodiment of the methods of treating, the administered T cells are prepared by a process comprising: collecting T cells from the human; and transforming the T cells with a recombinant vector ex vivo to generate recombinant T cells. In some embodiments, the method further comprises amplifying the recombinant T cells ex vivo.

Another embodiment provides a bifunctional protein comprising: a CD3-binding domain; and an HPV 16 E6-91-115-binding domain. The bifunctional protein comprises a single chain antibody fragment (single chain Fv) that comprises the CD3-binding domain and CDR3 segments of TCR alpha and TCR beta polypeptides that form at least a portion of the E6 91-115-binding domain.

In one more specific embodiment of the bifunctional protein, the protein comprises two polypeptides, (a) and (b), connected by a cysteine disulfide, wherein polypeptide (a) is a fusion polypeptide comprising a TCR alpha segment and a single chain Fv comprising the CD3-binding domain, and polypeptide (b) comprises a TRL beta segment, wherein the TCR alpha and TCR beta segments form the E6 91-115 binding domain

In a more specific embodiment, the bifunctional protein comprises (a) a TCR alpha CDR3 sequence SEQ ID NO:5 and a TCR beta CDR3 sequence SEQ ID NO:2; (b) a TCR alpha CDR3 sequence SEQ ID NO:6 or 7 and TCR beta CDR3 sequence SEQ ID NO:1; (c) a TCR alpha CDR3 sequence SEQ ID NO: and TCR beta CDR3 sequence SEQ ID NO:3; or (d) a TCR alpha CDR3 sequence SEQ ID NO:9 and TCR beta CDR3 sequence SEQ ID NO:4.

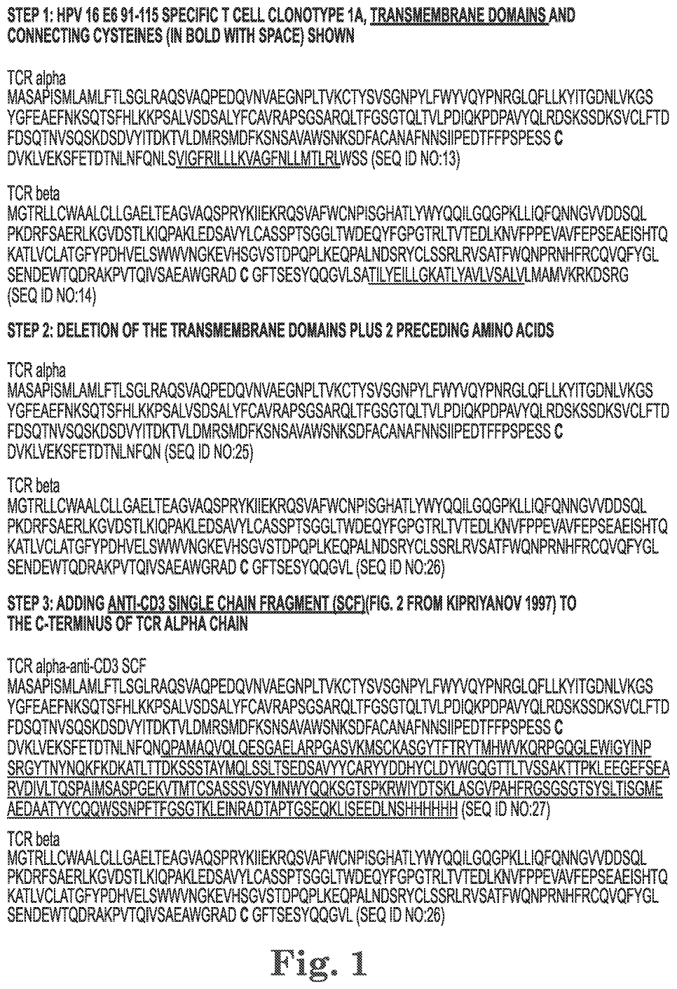

In a still more specific embodiment, the polypeptide (a) comprises SEQ ID NO:27 and polypeptide (b) comprises SEQ ID NO:26. (FIG. 1, described more fully in Example 2).

Another embodiment provides a method of treating a human for a cancer caused by HPV or other disease caused by HPV comprising: administering a bifunctional protein of the invention to a human afflicted with a cancer caused by HPV or other disease caused by HPV.

### EXAMPLE 1

### Expansion of Human Papillomavirus-Specific T cells in Periphery and Cervix in a Therapeutic Vaccine Recipient Whose Cervical High Grade Squamous Intraepithelial Lesion Regressed

Advances in high-throughput sequencing have revolutionized the manner with which we can study T cell responses. We describe a woman who received a human papillomavirus (HPV) therapeutic vaccine called PepCan, and experienced complete resolution of her cervical high-grade squamous intraepithelial lesion. By performing bulk T cell receptor (TCR) beta deep sequencing of peripheral blood mononuclear cells (PBMC) before and after 4 vaccinations, 67 significantly increased therefore putatively vaccine-specific TCRs were identified using a beta-binomial model. By combining information obtained from routinely performed enzyme-linked immunospot assay which detected the strongest HPV-specific T cell response in the HPV 16 E6 91-115 region after 4 vaccinations and single-cell RNA-seq and TCR sequencing of interferon-γ secreting HPV-specific T cells, the HPV specificity in 60 of these 70 TCRs were demonstrated. TCR β bulk deep sequencing of the cervical liquid-based cytology samples and cervical formalin-fixed paraffin-embedded samples before and after 4 vaccinations demonstrated the presence of these HPV-specific T cells in cervix. Combining traditional and cutting edge immunomonitoring techniques enabled us to demonstrate expansion of HPV-antigen specific T-cell in periphery and the cervix.

### Introduction

We evaluated the safety of an HPV therapeutic vaccine (PepCan) in a single center, single arm, dose-escalation Phase I clinical treating women with biopsy-proven HSILs (NCT01653249) (7,8). PepCan consists of four current good manufacturing practice (cGMP)-grade peptides covering the human papillomavirus type 16 (HPV 16) E6 protein (amino acids 1-45, 46-80, 81-115, and 116-158) and Candida albicans skin test reagent (Candin®, Nielsen Biosciences, San Diego, CA). PepCan was shown to be safe, resulted in histological regression rate of 45% which is roughly double that of a historical placebo (22%) (9). In addition, circulating T-helper type I (Th1) cells in periphery (p=0.0004) was increased, and the HPV 16 viral load was significantly decreased (p=0.008) (7).

Recent advances in high-throughput sequencing technology have enhanced our ability to appreciate how T cell receptor (TCR) repertoire may reveal the role of T cells in immunotherapy for HPV-related diseases (10-12). The actual diversity present in a human body is estimated to be around 10¹³ different TCRs (13). The next generation sequencing can facilitate the simultaneous analysis of millions of TCR sequences. Understanding the cytotoxic T cells repertoire, as well as a clinical response, would be essential for revealing immune mechanisms behind immunotherapies for chronic infectious diseases or cancer (10, 14-17). In this example, we utilize multiplexed PCR-based TCR sequencing using genomic DNA to characterize TCR repertoires in peripheral blood mononuclear cells (PBMCs), stimulated CD3 cells, formalin-fixed paraffin-embedded (FFPE) tissues, and liquid-based cytology (LBC) samples from one subject who was a histological responder from the Phase I clinical trial mentioned above. In addition, single cell RNA-seq and TCR sequencing approaches were utilized to reveal the TCR sequences of HPV-specific T cells with a specificity to the HPV 16 91-115 amino acid region revealed by the enzyme-linked immunospot (ELISPOT) assay. We provide a proof of principle that a traditional immune assay such as the ELISPOT assay can be combined with a cutting-edge technology to characterize HPV-specific T cells.

### Results

### Clinical trial design and vaccine response

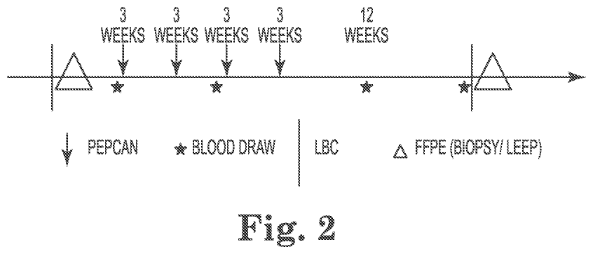

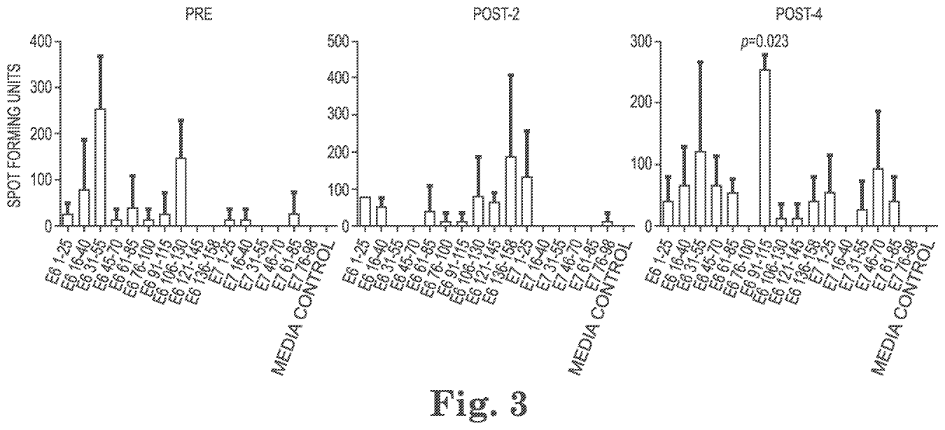

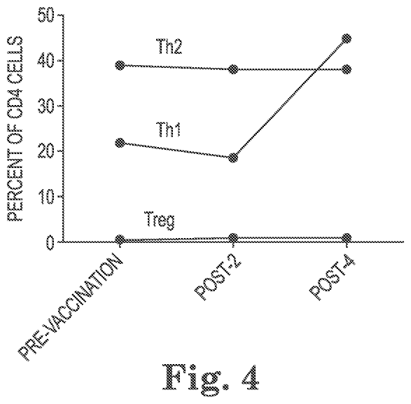

The subject participated in a single-arm an open-label Phase I clinical trial of HPV therapeutic vaccine, PepCan, treating women with biopsy-proven HSILs (FIG. 2) (7,8). This was a 41-year-old Caucasian woman, and she had cervical intraepithelial neoplasia 3 (CIN 3) at entry, and was positive for HPV types 16, 31, and 58. At study exit, her LEEP biopsy was benign but was noted to have marked lymphocytic infiltration. She was noted to have leukocytosis and lymphocytosis (Table 1), and was positive for HPV 40 at exit. ELISPOT assay showed pre-existing HPV-specific response and responses to multiple HPV regions detected after vaccination. The response to one region, HPV 16 E6 91-115, was significantly increased after four vaccinations (FIG. 3, p=0.023). Peripheral immune cell profiling showed increased percentage of Th1 cells, but unchanged levels of Tregs and Th2 cells (FIG. 4). Her HLA types were HLA-A*24/A*30, B*15/B*51, C*01/C*03, DPB1*02:01/DPB1*02:01, DQB1*03/DQB 1*06, and DRB1*11/DRB1*1.3.

TABLE 1
Complete blood count with differentials

| Test | Reference range | Pre | Post-2 | Post-4 |
|---|---|---|---|---|
| WBC (K/uL) | 3-12 | 7.92 | 8.14 | 13.94 |
| Hemoglobin (g/dL) | 11.5-16 | 12.9 | 13.4 | 13.9 |
| Hematocrit (%) | 34-47 | 39.1 | 41.2 | 42.7 |
| Platelet (K/uL) | 150-500 | 225 | 225 | 237 |
| Neutrophils (K/uL) | 2.5-8.2 | 4.5 | 5 | 7 |
| Lymphocyte (K/uL) | 1-4.8 | 2.5 | 2.3 | 5.5 |
| Monocytes (K/uL) | 0.1-1 | 0.6 | 0.6 | 0.9 |
| Eosinophils (K/uL) | 0-0.4 | 0.3 | 0.2 | 0.4 |
| Basophils (K/uL) | 0-0.22 | 0.02 | 0.02 | 0.03 |

Bold indicates values outside of the reference range.

### Multiplexed PCR-based TCR β Chain Deep Sequencing

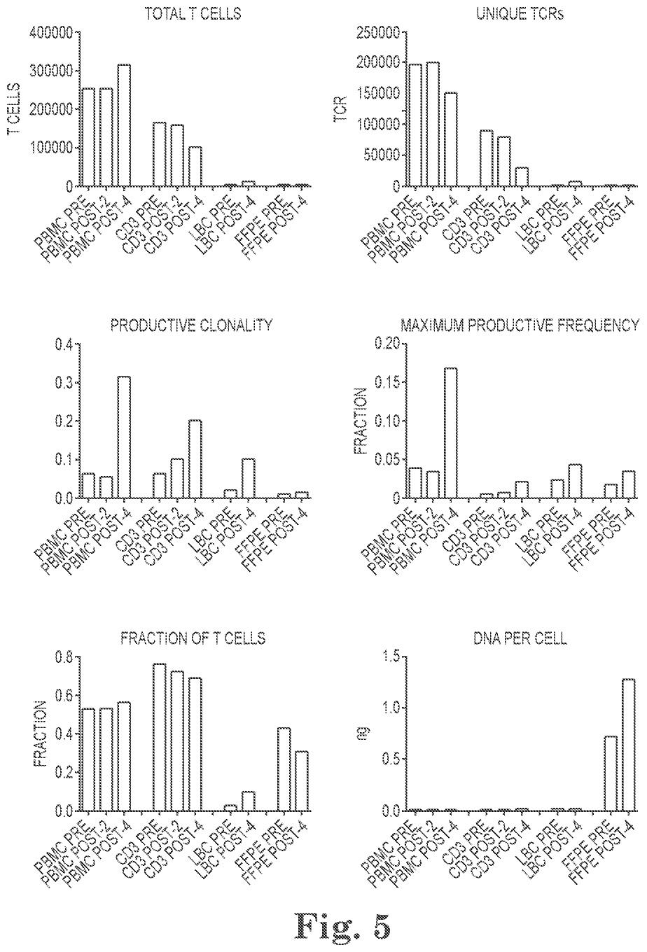

All samples examined (n=10, PBMC and stimulated CD3 samples at pre, post-2, and post-4, and PEPE and LBC samples at pre and post-4) types yielded sufficient. quantities of DNA for bulk TCR sequencing. In total, 749,417 clonotypes, and 1,256,277 T cells were identified in these 10 samples (Table 2). The numbers of total T cells and clonotypes were higher in PBMC than in stimulated CD3 (FIG. 5). In cervical samples, the clonotypes (372, 344, 699 and 6,748) and total T cells (403, 382, 814, and 10,731) were detected in FFPE (pre and post-4) and LBC (pre and post-4). The productive clonality was increased after 4 vaccination in PBMC, stimulated CD3 and LBC samples, and the maximum productive frequencies at least doubled in all sample types (FIG. 5). The T cell fraction was the highest in stimulated CD3 cells, and lowest in LBCs. DNA per cell was similar among PBMC, stimulated CD3 cells, and LBC (ranging from 0.0061 ng per cell to 0.011 ng per cell), but much higher in the FFPE, samples (0.714 ng per cell for pre and 1.27 ng per cell for post-4) possibly reflecting lower quality DNA.

TABLE 2
Sample characteristics.

| | | Used | Input | | Clonotypes | | Clonotypes |
|---|---|---|---|---|---|---|---|
| Sample | Vaccines | sample | DNA | T cells by | by | T cells by | by amino |
| types | time | amount | (ng) | nucleotides | nucleotides | amino acids | acids |
| PBMC | Pre | 8 × 10⁶ | 2,852 | 252,926 | 195,744 | 252,926 | 187,972 |
| | | cells | | | | | |
| | Post-2 | 8 × 10⁶ | 2,861 | 253,155 | 199,650 | 253,155 | 191,481 |
| | | cells | | | | | |
| | Post-4 | 8 × 10⁶ | 3,428 | 313,245 | 149,604 | 313,245 | 144,519 |
| | | cells | | | | | |
| Stimulated | Pre | 6.8 × 10⁶ | 1,204 | 166,173 | 88,391 | 166,173 | 85,767 |
| CD3 | | cells | | | | | |
| | Post-2 | 6.5 × 10⁶ | 1,202 | 158,747 | 78,715 | 158,747 | 76,644 |
| | | cells | | | | | |
| | Post-4 | 2 × 10⁶ | 1,202 | 99,701 | 29,150 | 99,701 | 28,643 |
| | | cells | | | | | |
| LBC | Pre | 1,200 μL | 318 | 814 | 699 | 814 | 694 |
| | Post-4 | 800 μL | 930 | 10,731 | 6748 | 10,731 | 6,693 |
| FFPE | Pre | Five 5 μ | 392 | 403 | 372 | 403 | 359 |
| | | scrolls | | | | | |
| | Post-4 | Five 5 μ | 934 | 382 | 344 | 382 | 331 |
| | | scrolls | | | | | |

T cell clone abundances were counted using complementarity determining region 3 (CDR3) nucleotide or amino acid sequences. PBMC: peripheral blood mononuclear cells, CD3: CD3 stimulated with HPV16 E6 and E7 antigens, LBC: liquid-based cytology, FFPE: formalin-fixed paraffin-embedded.

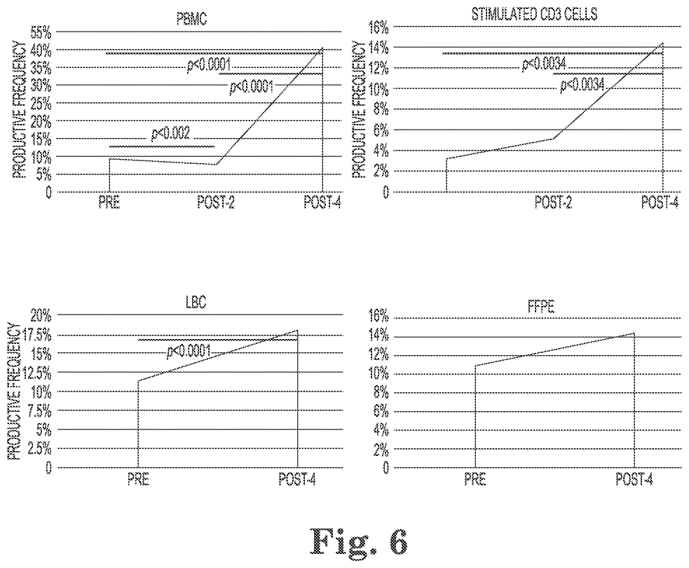

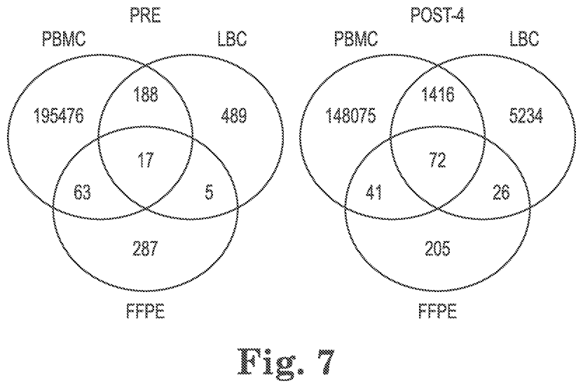

The percentages of the top 15 most frequent clonotypes were significantly increased after 4 vaccination in all sample types except for FFPE (FIG. 6). Venn diagrams of PBMC, LBC, and FFPE at pre-vaccination and post-4 vaccination respectively revealed that some clonotypes can be detected in all sample types reflecting the capacity of at least a subset of T cells to traffic to the cervix (FIG. 7). A beta-binomial model which accounts for variance due to random sampling from a highly diverse repertoire and time-dependent variance for identifying clinically relevant expansion of T cells 18 was used to identify putatively vaccine-specific TCRs using pre and post-4 PBMC samples. Seventy putatively vaccine-specific TCRs were identified using the CDR3 nucleotide sequences. The numbers of such clonotypes and total T cells in pre and post-4 vaccination FFPE (1 and 9 clonotypes, and 1 and 13 total T cells respectively) and pre and post-4 vaccination LBC [14 and 47 clonotypes, and 33 and 1,523 total T cells respectively] showed that LBC may be more an informative sample type because of T cell abundance.

### Single-cell RNA-seq and TCR Sequencing

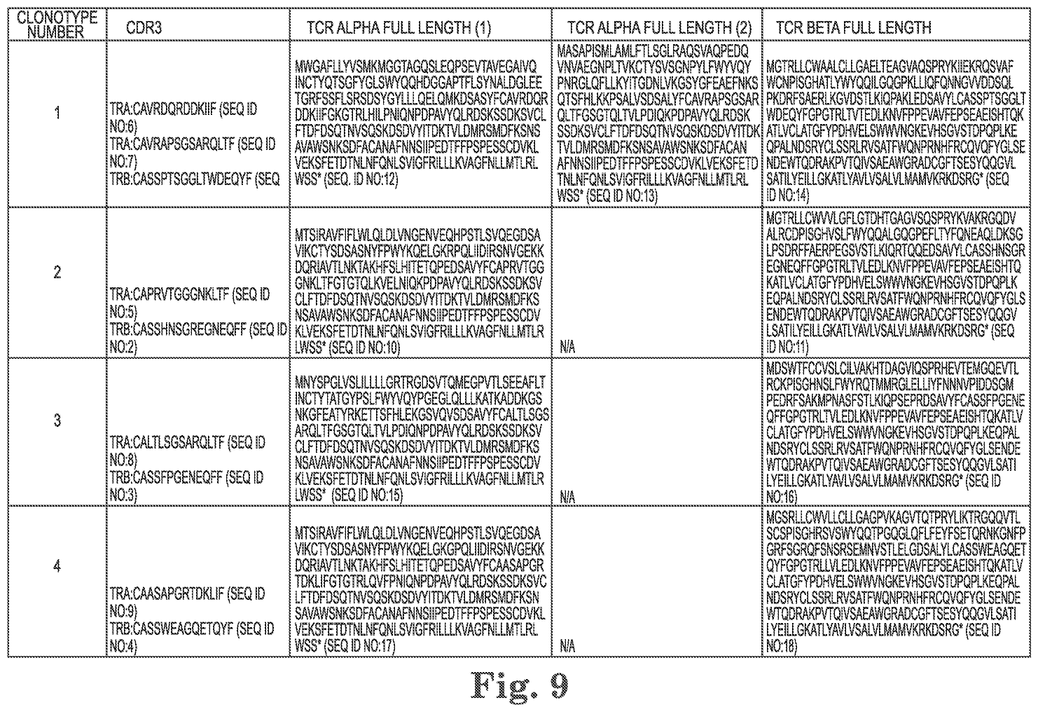

Of 8.5×10⁶ peptide-stimulated and labeled cells, 1.3×10⁶ (15.3%) were positively sorted for secreting IFN-γ. For the TCR sequencing, the estimated number of cells were 12,240 with the mean read pairs of 13,678 per cell. Most (10,246 of 12,240 or 83.7%) cells contained productive V-J spanning pairs. The TCR β amino acid sequences of the 4 clonotypes with a frequency of ≥5% among the IFN-γ positive cells are shown in Table 3 and the TCR alpha CDR3 sequences and full length TCRA and TCRB polypeptides are shown in FIG. 9.

TABLE 3
TCR β CDR3 sequences of clonotypes with specifi-
city to HPV 16 E6 91-115 and ≥5% frequency.

| Clono- | | Frequency | |
|---|---|---|---|
| type | Number | (%) | Amino acid sequence |
| 1 | 2,615 | 33.3 | CASSPTSGGLTWDEQYF |
| | | | (SEQ ID NO: 1) |
| 2 | 1,340 | 17.0 | CASSHNSGREGNEQFF |
| | | | (SEQ ID NO: 2) |
| 3 | 772 | 9.8 | CASSFPGENEQFF |
| | | | (SEQ ID NO: 3) |
| 4 | 678 | 8.6 | CASSWEAGQETQYF |
| | | | (SEQ ID NO: 4) |

The single-cell RNA-seq analysis revealed 15,114 estimated number of cells, 32,659 mean reads per cell, and 2,047 median number of genes per cell. The representation of the total unique molecular identifiers (UMI) counts for each cell-barcode reveal various level of gene expression among the IFN-γ producing cells, and clustering showed IFN-γ mRNA expression only in a minority of cells belonging to cluster 13.

### Tracking of the HPV 16 E6 91-115 Specific T Cells

Using the TCR R CDR3 sequences of the 4 clonotypes specific for HPV 16 E6 91-115, their frequencies in PBMC, LBC, and FFPE samples were determined using the TCR β chain deep sequencing (FIG. 5). All 4 clonotypes were represented in the top 15 most frequent clonotypes for PBMCCs, LBC, and stimulated CD3+ T cells, but only clonotype 1 was present in FFPE (FIG. 6). Of the 70 clonotypes identified to be putatively vaccine-specific using the beta-binomial model, 60 clonotypes were shown to be HPV 16 E6 91-115 specific. Clonotype 1 was the most abundant clonotype stimulated CD3+ T cells.

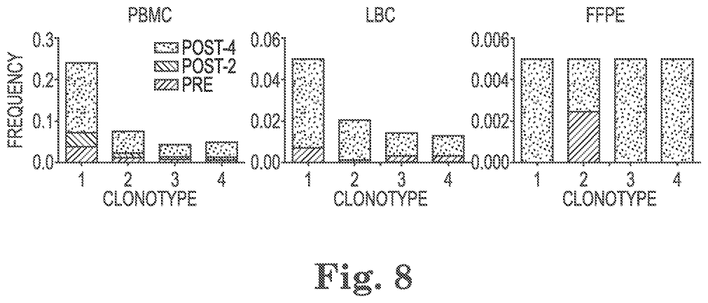

All 4 clonotypes were detectable in PBMCs and LBC prior to vaccination, and their expansion after 4 vaccinations is shown (FIG. 8). Only one T cell of clonotype 2 is detectable prior to vaccination in FFPE. All 4 clonotypes were detectable after 4 vaccinations, but only at 2 T cells for clonotypes 1, 3, and 4, and 1 T cell for clonotype 2 (FIG. 8). As much fewer cells were detected in FFPE, LBC was a better source for assessing T cell populations, at least in this subject.

We tracked HPV 16 E6 91-115 specific T cells in PBMC, LBC, and FFPE. The TCR Vα and Vβ sequences of HPV 16 E6 91-115 specific T cells were determined by sorting and sequencing such cells based on IFN-gamma secretion upon peptide stimulation. The TCR Vβ CDR3 sequences of top 4 clonotypes (≥5% of IFN-γ secreting cells) are shown in Table 3. The frequencies of these clonotypes in PBMC, LBC, and FITE at pre-vaccination, post-2 vaccinations, and post-4 vaccinations time points are shown in FIG. 8. All 4 vaccine-specific clonotypes PBMC and LBC increased in frequency after 4 vaccinations. On the other hand, data from FFPE were not as informative.

### Discussion

This was a proof of concept study to demonstrate the utility of TCR analyses using high throughput sequencing technology in a context of HPV therapeutic vaccine trials. The earliest evidence of the link between HPV and cervical cancer was discovered in 1983 by Harald zur Hausen and his colleagues (19). A Nobel Prize was later awarded. To date, over 200 HPV types have been described (20). HPV antigens are ideal targets for cancer immunotherapy because they are foreign. Various versions of investigational HPV therapeutic vaccines have been in clinical trials for about the last 30 years, but none has been approved by the United States Food and Drug Administration (FDA). Investigational HPV therapeutic vaccines have been tested for many indications including clearance of HPV 16 and/or 18 infection (21), HSIL regression (7,8,22) prevention of recurrence of squamous cell carcinoma of head and neck (HNC) (23), treatment of advanced stage cervical cancer (24,25), and treatment of advanced stage HNC (26). The assessment of vaccine efficacy depends on the indication being tested. For HPV 16 infection clearance, HPV-DNA typing was used (21), and biopsies are utilized to evaluate HSIL regression (7,8,22). Lack of recurrence within a 2 year period is being used for assessing prevention of recurrence (23). Antitumor efficacy was examined using the numbers of patients with complete and partial response, tumor shrinkage, duration of response (25).

Unlike the HPV prophylactic vaccines which work by inducing production of neutralizing antibodies (27,28), the HPV therapeutic vaccines are believed to cast their effects through stimulation of cell-mediated immunity, mainly the T cells. Therefore, the assessments of T cell immune responses should be included in the endpoints of clinical trials. However, such implementation varies widely among the clinical trials because T cell assays are technically challenging. In Maciag et al. (24), the investigators attempted to perform IFN-γ ELISPOT assay using pooled peptides, but most samples were not suitable after thawing due to low yield and viability. Of the 3 patients with sufficient amount of cells available to perform the assay, only one demonstrated HPV-specific T cell response after vaccination. HPV 16 E7 short and long peptides were pooled before testing, so no information as to which portion of the protein contained immunogenic epitopes was obtained (24). In the GTL001 trial, van Domme et al. performed ex vivo IFN-γ ELISPOT assay with pooled HPV 16 E7 peptides or HPV 18 E7 peptides. Overall, 18 of 31 (58.1%) of patients who received any dose of GTL001 with imiquimod demonstrated positive ELISPOT results to either protein. Trimble et al. also tested immune responses using IFN-γ ELISPOT assay and intracellular cytokine staining for assessment of T cell immunity. Significantly higher responses were reported for patients who received the VGX-3100 vaccine compared to those who received placebo. As peptides were pool for each protein tested (HPV 16 E6, HPV 16 E7, HPV 18 E6, and HPV 18 E7), information on which portion of the protein contained immunogenic epitopes were not determined (22). In the clinical trial which treated patients with advance HNC with ISA100 and nivolumab, the investigators performed IFN-γ ELISPOT assay for HPV 16 E6 and E7 again using peptide pools. Although variable increases in the number of HPV-specific T cells were observed after vaccination in both responders and nonresponders. Furthermore, the immune response did not correlate with efficacy endpoints (26). In addition to IFN-γ ELISPOT assay, Melief et al. performed lymphocyte stimulation test, intracellular cytokine staining, and cytometric bead array to assess immune responses. In all 64 patients who received ISA101 vaccination, HPV 16 E6 and/or E7-specific T cell responses to one or more of 6 peptide pools (4 pools for HPV 16 E6 and 2 pools for HPV 16 7 protein) was demonstrated. Our IFN-γ ELISPOT protocol distinguishes itself among others in that we tested for 10 HPV 16 E6 peptides pools and 6 HPV 16 E7 peptide pools (FIG. 3) (7,8). Therefore, the locations of the antigenic epitopes can be narrowed to 25 amino acid regions. This characteristic of our assay was a key to identify a significant response to the HPV 16 E6 91-115 region, and subsequent isolation of T cells based on IFN-γ secretion. Overall, 19 of 31 (61%) of vaccine recipients demonstrated new T cell response to at least one region of HPV 16 E6 protein. Furthermore, the responses were statistically significant in 13 of 31 (42%) of subjects. However, such responses observed after vaccination in periphery did not correlate with whether the cervical HSIL lesions regressed. It is likely that the vaccine-induced HPV-specific T cells failed to reach the cervix in some subjects.

TCRs are highly diverse heterodimers consisting of α and β chains in the majority of T cells. However, 1 to 5% of T cells express γδ chains (29). Similar to B cell receptors, the TCR chains contains a variable region responsible for antigen recognition, and a constant region. The variable region of the α and δ chains is encoded by recombined variable (V) and joining (J) genes. Additionally for the β and γ chains, diversity (D) genes are also recombined (i.e., VDJ recombination). Therefore, the β and γ chains are more diverse than the α and δ chains. The advent of high throughput sequencing made it possible to probe into the complexity of such TCRs. In this currently study, we employed TCR β chain deep sequencing using bulk DNA and single-cell RNA based TCR analysis using mRNA. The former has the advantage of using DNA which can be extracted from LBC and FFPE samples; therefore, live cells are not necessary. The latter was utilized to analyze IFN-γ secreting HPV 16 E6 91-115 specific T cells. Not only information on TCR α and β sequences and their parings, but also gene expression profile of individual cells were examined We demonstrated that using the information from a traditional IFN-γ ELISPOT assay in combination with TCR sequencing enable us to demonstrate the expansion of HPV-specific T cells and their presence in the cervix. In addition for gaining the information on TCR α and β chain pairings, the single-cell RNA based method has the advantage of yielding the sequences of the α and β chains. This would enable construction of the TCRs in viral rectors with which their specificities can be verified,{Lu, 2018 #685;Morgan, 2006 #686} but such engineered T cells can be used for immunotherapy as demonstrated by Draper and colleagues {Draper, 2015 #644}. T cells genetically engineered to express the TCR of HPV 16 E6 29-38 (TIHDIILECV (SEQ ID NO:19) epitope restricted by HLA-A*02:01 were shown to be cytotoxic for HPV 16 positive cervical, and head and neck cancer cell lines. The limitation of this study was that we only examined one subject in a proof of concept study. As the Phase II clinical trial of PepCan is ongoing (NCT02481414), additional analyses of Phase II participants would aid in determining the generalizability of the findings of this study.

### Ethics

### Methods

This study was approved by the Institutional Review Board at the University of Arkansas for Medical Sciences (IRB number 130662) and written informed consent was obtained.

### Subject, Clinical Trial Design, and Laboratory Analyses

This open-label single center dose-escalation Phase I clinical trial of PepCan was reported previously (7,8). Subject 6 was selected for the current study because she was a vaccine responder, and sufficient amounts of her samples were available for further analyses. Briefly, subjects qualified for vaccination if biopsy-proven CIN 2 and/or CIN 3 (FIG. 2). PepCan (subject 6 received 50 μg/peptide dose) was given 4 times 3 weeks apart, and LEEP was performed 1:2 weeks after the last vaccination. Cervical liquid-based cytology (LBC) samples (ThinPrep, Hologic, Marborough, MA) were collected for HPV typing before vaccination at the time of qualifying biopsy, and after 4 vaccinations at the time of LEER Blood was drawn before vaccination, after 2 vaccinations, and after 4 vaccinations, and routine clinical laboratory tests (complete blood count, sodium, potassium, chloride, carbon dioxide, blood urea nitrogen, creatinine, aspartate transaminase, alanine transaminase, lactate dehydrogenase, 7-glutamyl transpeptidase, total bilirubin, and direct bilirubin) were, performed. PBMC were isolated using ficoll density gradient method. Cells were stored in liquid nitrogen tanks while LBC samples were kept in −80° C. freezers. FFPE samples were stored at room temperature.

Research laboratory analyses performed as a part of the clinical trial included HPV typing (Linear Array HPV Genotyping Test, Roche Molecular Diganostics, Pleasanton, CA), IFN-γ ELISPOT assay, fluorescent-activated cell sorter analysis of peripheral Th1, Th2, and Treg cells, and HLA class I and class II low-resolution typing (One Lambda, West Hills, CA). The Linear Array HPV Genotyping Test detects 37 individual HPV types (6, 11, 16, 18, 26, 31, 33, 35, 39, 40, 42, 45, 51, 52, 53, 54, 55, 56, 58, 59, 61, 62, 64, 66, 67, 68, 69, 70, 71, 72, 73, 81, 82, 83, 84, IS 39, and CP6108). For the ELISPOT assay, magnetically selected CD3 T cells were stimulated with autologous monocyte-derived dendritic cells pulsed with HPV 16 E6 or E7 using recombinant vaccinia viruses and recombinant GST fusion proteins twice for one week duration for each stimulation. The assay was performed in triplicates using overlapping HPV 16 E6 and E7 peptides covering HPV 16 E6 1-25, 16-40, 31-55, 45-70, 61-85, 76-100, 91-115, 106-130, 121-145, 136-158 and HPV 16 E7 16-40, 31-55, 46-70, 61-85, and 76-98 regions, PBMCs were stained for CD4, CD25, T-bet, GATA3, and Foxp3. The percentage of CD4 cells positive for T-bet represented Th1 cells, those positive for GATA3 represented Th2 cells, and those positive for CD25 and FoxP3 represented Tregs.

### Multiplexed PCR-based TCR Sequencing

The TCRβ CDR3 regions were PCR-amplified and sequenced (immunoSEQ, Adaptive Biotechnologies, Seattle, WA (30)) using genomic DNAs from PBMC (pre, post-2, and post-4), stimulated CD3 T cells (pre, post-2, and post-4), LBC (pre and post-4), and FFPE (pre and post-4). Using bias-controlled V and J gene primers, the rearranged V(D)J segments were amplified and sequenced. A clustering algorithm was used to correct for sequencing errors, and the CDR3 segments were annotated according to the International ImMunoGeneTicsCollaboration (31,32) to identify the V, D, and J genes that contributed to each rearrangement. A mixture of synthetic TCR analogs was used in PCR to estimate the number of cells bearing each unique TCR sequence (33) “Detailed rearrangements”, “Track Rearrangements”, “Venn Diagram”, “Differential Abundance”, and “Scatterplot with Annotation” features of the immunoSeq analyzer³⁴ were used to analyze data.

### Single-cell RNA-seq of HPV-specific T Cells

In order to obtain TCR Vα and VIβ sequences of T cells specific for HPV 16 E6 91-115, such T cells were selected using a human IFN-γ Secretion Assay—Cell Enrichment and Detection Kit (Miltenyi Biotec, Auburn, CA) following the manufacturer's instructions as previously described.³⁵⁻³⁹ Post-4 PBMC sample cryopreserved after monocyte depletion was thawed and cultured overnight in Yssel's media (Gemini Bio Products, West Sacramento, CA) with 1% human serum and 1,200 IU/ml of recombinant human interleukin-2 (R&D Systems, Inc., Minneapolis, MN). As a positive control, healthy donor PBMC mixed with 1% HPV 16 E6 52-61 (FAFRDLCIVY (SEQ ID NO:20))-specific T cell clone cells was processed in the same manner The cells were stimulated for 3 h with 10 μM each of peptides in RPMI1640 media plus 5% human serum: FAFRDLCIVY (SEQ ID NO:20) for the positive control, and the three 15-mer overlapping peptides covering the E6 91-115 region (91-105, YGTTLEQQYNKPLCD (SEQ ID NO:21); 96-110, EQQYNKPLCDLLIRC (SEQ ID NO:22); 101-115, KPLCDLLIRCINCQK (SEQ ID NO:23) for subject 006. IFN-γ secreting cells were labeled using the IFN-γ catch reagent and phycoerythrin (PE) labeled IFN-γ detection antibody. The positive control sample and healthy donor PBMC stained with mouse IgG1K isotype labeled with PE (eBiosciences) was used as a negative control to set the gate. The IFN-γ positive cells were sorted using FACS Aria (BD Biosciences, Franklin Lakes, New Jersey.

A Next GEM Chip G was loaded with approximately 10,000 cells and Chromium Next GEM Single Cell 5′ Library Gel Bead Kit v1.1 reagent. An emulsion was generated with the Chromium Controller (10× Genomics). Gene expression (GEX) libraries were prepared with the Chromium Single Cell 5′ Library Construction Kit and TCR libraries were prepared with the Chromium Single Cell V(D)J Enrichment Kit, Human T Cell (10× Genomics). A low-pass surveillance sequencing run of both libraries were performed on separate Illumina mid-output MiniSeq flow cells (GEX library Readl1:26bp, Read2:91bp, TCR library Read1:150bp, Read2:150bp). Sequencing was scaled up on an Illumina NextSeq 500 with a high-output 150-cycle v2.5 kit for the GEX library and a mid-output 300-cycle v2.5 kit for the TCR library; both runs used identical read lengths as on the MiniSeq. Data was aggregated from both runs.

Sequencing data were first processed by a Cell Ranger pipeline (v4.0.0; 10× Genomics). Gene expression sequencing data were mapped to human reference (GRCh38-2020A) dataset, and TCR sequencing data were mapped to human TCR reference (GRCh38-alts-ensembl-4.0.0) dataset. Gene expression profiles were further analyzed by Loupe Browser (v4.2.0; 10× Genomics) and TCR data were further analyzed by Loupe V(D)J Browser (v3.0.0; 10× Genomics). T-cell clonotypes were defined based on TCR VEβ CDR3 nucleotide sequences after removing single cells containing only α chains and containing two different TCR VEβ CDR3 nucleotide sequences (likely doublets). For calculating the frequencies of ≥5% clonotypes (Table 3), single cells with two or more copies were included. Full-length TCRα/β amino acid sequences were obtained by the Loupe V(D)J Browser.

### Data Availability

The bulk TCR VP deep sequencing data are available in immuneACCESS (immuneACCESS DOL https://DOI10.21417/TS2020HPV).

### Statistical Analysis

A paired t-test was performed to assess the significant changing of spot forming unis (i.e., IFN-γ secreting cells) before and after the vaccines in ELISPOT assay. The number of T cells between study visits in PBMC, stimulated CD3 T cells, LBC, and FFPE were compared using Wilcoxon matched-pairs singed-ranks sum test (GraphPad Instat 3, GraphPad Software, San Diego, CA). A p value <0.05 was considered statistically significant.

### HPV 16 E6 Protein Sequence

(SEQ ID NO: 24)
MHQKRTAMFQ DPQERPRKLP QLCTELQTTI HDIILECVYC
KQQLLRREVY DFAFRDLCIV YRDGNPYAVC DKCLKFYSKI
SEYRHYCYSL YGTTLEQQYN KPLCDLLIRC INCQKPLCPE
EKQRHLDKKQ RFHNIRGRWT GRCMSCCRSS RTRRETQL

### EXAMPLE 2

A bispecific protein that has moieties that bind HPV 16 E6 91-115 and CD3 in order to direct T cells (with the CD3 target) to HPV-infected cells (with the E6 91-115 target). The anti-CD3 portion is provided in this example by a single chain Fv antibody arm from the biologic Tebentafusp (40). The anti-E6-91-115 is provided by the domain formed by TCRA of SEQ ID NO:13 and the TCRB of SEQ ID NO:14 in Clonotype 1. As shown in FIG. 1, the transmembrane segments and 2 residues to the N terminus of them are deleted to produce TCR alpha SEQ ID NO:25 and TCR beta SEQ ID NO:26. The anti-CD3 single chain fragment is fused at its N terminus to the C terminus of TCR alpha SEQ ID NO:25 to produce TCR alpha-anti-CD3 SCF fusion protein SEQ ID NO:27. The SEQ ID NO:27 and soluble abbreviated TCR beta of SEQ I ID NO:26 can be expressed in E. coli and refolded and linked by a disulfide formed between the bolded cysteines by standard techniques and as described in (41,42).

### References

1. HPV-Associated Cancer Statistics. Vol. 2019 (Centers for Disease Control and Prevention, 2019).

### 2. Cancer Facts & FIGS. 2019. (American Cancer Society, 2019).

3. de Martel, C., Plummer, M., Vignat, J. & Franceschi, S. Worldwide burden of cancer attributable to HPV by site, country and HPV type. Int J Cancer 141, 664-670 (2017).
4. Hildesheim, A., et al. Effect of human papillomavirus 16/18 L1 viruslike particle vaccine among young women with preexisting infection: a randomized trial. JAMA 298, 743-753 (2007).
5. Massad, L. S., et al. 2012 updated consensus guidelines for the management of abnormal cervical cancer screening tests and cancer precursors. Obstet Gynecol 121, 829-846 (2013).
6. Bruinsma, F. J. & Quinn, M. A. The risk of preterm birth following treatment for precancerous changes in the cervix: a systematic review and meta-analysis. BJOG 118, 1031-1041 (2011).
7. Coleman, H. N., et al. Human papillomavirus type 16 viral load is decreased following a therapeutic vaccination. Cancer Immunol Immunother 65, 563-573 (2016).
8. Greenfield, W. W., et al. A phase I dose-escalation clinical trial of a peptide-based human papillomavirus therapeutic vaccine with skin test reagent as a novel vaccine adjuvant for treating women with biopsy-proven cervical intraepithelial neoplasia 2/3. Oncoimmunology 4, e1031439 (2015).
9. Nieminen, P., et al. Efficacy and safety of R05217990 treatment in patients with high grade cervical intraepithelial neoplasia (CIN2/3). in 28th International Papillomavirus Conference (Puerto Rico, 2012).
10. Cui, J. H., et al. TCR Repertoire as a Novel Indicator for Immune Monitoring and Prognosis Assessment of Patients With Cervical Cancer. Front Immunol 9, 2729 (2018).
11. Lang Kuhs, K. A., et al. T cell receptor repertoire among women who cleared and failed to clear cervical human papillomavirus infection: An exploratory proof-of-principle study. PLoS One 13, e0178167 (2018).
12. Morrow, M. P., et al. Augmentation of cellular and humoral immune responses to HPV16 and HPV18 E6 and E7 antigens by VGX-3100. Mol Ther Oncolytics 3, 16025 (2016).
13. Laydon, D. J., Bangham, C. R. & Asquith, B. Estimating T-cell repertoire diversity: limitations of classical estimators and a new approach. Philos Trans R Soc Lond B Biol Sci 370(2015).

### 14. Fink, K. Can We Improve Vaccine Efficacy by Targeting T and B Cell Repertoire Convergence? Front Immunol 10, 110 (2019).

15. Hopkins, A. C., et al. T cell receptor repertoire features associated with survival in immunotherapy-treated pancreatic ductal adenocarcinoma. JCI Insight 3(2018).
16. Inoue, H., et al. Intratumoral expression levels of PD-L1, GZMA, and HLA-A along with oligoclonal T cell expansion associate with response to nivolumab in metastatic melanoma. Oncoimmunology 5, e1204507 (2016).
17. Tumeh, P. C., et al. PD-1 blockade induces responses by inhibiting adaptive immune resistance. Nature 515, 568-571 (2014).
18. Rytlewski, J., et al. Model to improve specificity for identification of clinically-relevant expanded T cells in peripheral blood. PLoS One 14, e0213684 (2019).
19. Durst, M., Gissmann, L., Ikenberg, H. & zur Hausen, H. A papillomavirus DNA from a cervical carcinoma and its prevalence in cancer biopsy samples from different geographic regions. Proc Natl Acad Sci U S A 80, 3812-3815 (1983).
20. Schiffman, M., et al. Carcinogenic human papillomavirus infection. Nat Rev Dis Primers 2, 16086 (2016).
21. Van Damme, P., et al. GTL001, A Therapeutic Vaccine for Women Infected with Human Papillomavirus 16 or 18 and Normal Cervical Cytology: Results of a Phase I Clinical Trial. Clin Cancer Res 22, 3238-3248 (2016).
22. Trimble, C. L., et al. Safety, efficacy, and immunogenicity of VGX-3100, a therapeutic synthetic DNA vaccine targeting human papillomavirus 16 and 18 E6 and E7 proteins for cervical intraepithelial neoplasia 2/3: a randomised, double-blind, placebo-controlled phase 2b trial. Lancet (2015).
23. ClinicalTrials.gov. Vol. 2014.
24. Maciag, P. C., Radulovic, S. & Rothman, J. The first clinical use of a live-attenuated Listeria monocytogenes vaccine: a Phase I safety study of Lm-LLO-E7 in patients with advanced carcinoma of the cervix. Vaccine 27, 3975-3983 (2009).
25. Melief, C. J. M., et al. Strong vaccine responses during chemotherapy are associated with prolonged cancer survival. Sci Transl Med 12(2020).
26. Massarelli, E., et al. Combining Immune Checkpoint Blockade and Tumor-Specific Vaccine for Patients With Incurable Human Papillomavirus 16-Related Cancer: A Phase 2 Clinical Trial. JAMA Oncol 5, 67-73 (2019).
27. Godi, A., et al. Durability of the neutralizing antibody response to vaccine and non-vaccine HPV types 7 years following immunization with either Cervarix(R) or Gardasil(R) vaccine. Vaccine 37, 2455-2462 (2019).
28. Sankaranarayanan, R., et al. Immunogenicity and HPV infection after one, two, and three doses of quadrivalent HPV vaccine in girls in India: a multicentre prospective cohort study. Lancet Oncol 17, 67-77 (2016).
29. Lo Presti, E., Dieli, F. & Meraviglia, S. Tumor-Infiltrating gammadelta T Lymphocytes: Pathogenic Role, Clinical Significance, and Differential Programing in the Tumor Microenvironment. Front Immunol 5, 607 (2014).
30. Robins, H. S., et al. Comprehensive assessment of T-cell receptor beta-chain diversity in alphabeta T cells. Blood 114, 4099-4107 (2009).
31. Lefranc, M. P., et al. IMGT(R), the international ImMunoGeneTics information system(R) 25 years on. Nucleic Acids Res 43, D413-422 (2015).
32. Yousfi Monod, M., Giudicelli, V., Chaume, D. & Lefranc, M. P. IMGT/JunctionAnalysis: the first tool for the analysis of the immunoglobulin and T cell receptor complex V-J and V-D-J JUNCTIONs. Bioinformatics 20 Suppl 1, i379-385 (2004).
33. Wu, D., et al. Detection of minimal residual disease in B lymphoblastic leukemia by high-throughput sequencing of IGH. Clin Cancer Res 20, 4540-4548 (2014).

### 34. Biotechnologies, A.

35. Coleman, H. A., Wang, X., Greenfield, W. W. & Nakagawa, M. A Human Papillomavirus Type 16 E6 52-62 CD4 T-Cell Epitope Restricted by the HLA-DR11 Molecule Described in an Epitope Hotspot. MOJ Immunology 1, 00018 (2014).
36. Nakagawa, M., Kim, K. H., Gillam, T.M. & Moscicki, A. B. HLA class I binding promiscuity of the CD8 T-cell epitopes of human papillomavirus type 16 E6 protein. J Virol 81, 1412-1423 (2007).
37. Nakagawa, M., Kim, K. H. & Moscicki, A. B. Different methods of identifying new antigenic epitopes of human papillomavirus type 16 E6 and E7 proteins. Clin Diagn Lab Immunol 11, 889-896 (2004).
38. Wang, X., Greenfield, W. W., Coleman, H. N., James, L. E. & Nakagawa, M. Use of Interferon-gamma Enzyme-linked Immunospot Assay to Characterize Novel T-cell Epitopes of Human Papillomavirus. J Vis Exp (2012).
39. Wang, X., Santin, A. D., Bellone, S., Gupta, S. & Nakagawa, M. A novel CD4 T-cell epitope described from one of the cervical cancer patients vaccinated with HPV 16 or 18 E7-pulsed dendritic cells. Cancer Immunol Immunother 58, 301-308 (2009).
40. Kipriyanov, S M, Moldenhauer, G, Martin, A C R, Kupryanova, O A, & Little, M. Two amino acid mutations in an anti-human CD3 single chain Fv antibody fragment that affect the yield on bacterila secretion but not the affinity. Protein Eng. 10:445-453 (1997).
41. Boudousquie, C, Bossi, G. et al. Polyfuncitonal response by ImmTAC (IMCgp100) redireceted CD8+ and CD4+ T cells. Immunology 152:425-438 (2017).
42. Boulter, J M, Glick M, et al. Stabel, soluble T-cell receptor molecules for crystalization and therapeutics. Protein Eng. 16:707-711 (2003).

The sequence listing file name 110-051US1.xml, size 28 kb, created August 14, 2023, is incorporated by reference.

What is claimed is:

1. A plurality of isolated recombinant human T cells enriched for recombinant human T cells comprising a recombinant T cell receptor beta (TCRB) gene encoding CDR3 sequence CASSPTSGGLTWDEQYF (SEQ ID NO:1) or CASSHNSGREGNEQFF (SEQ ID NO:2) or CASSFPGENEQFF (SEQ ID NO:3) or CASSWEAGQETQYF (SEQ ID NO:4).

2. The isolated recombinant human T cells of claim 1 comprising: (a) a recombinant T cell receptor alpha (TCRA) gene encoding CDR3 sequence SEQ ID NO:5 and a T cell receptor beta (TCRB) gene encoding CDR3 sequence SEQ ID NO:2; (b) a recombinant TCRA gene encoding CDR3 sequence SEQ ID NO:6 or 7 and a recombinant TCRB gene encoding CDR3 sequence SEQ ID NO:1; (c) a recombinant TCRA gene encoding CDR3 sequence SEQ ID NO:8 and a recombinant TCRB gene encoding CDR3 sequence SEQ ID NO:3; or (d) a recombinant TCRA gene encoding CDR3 sequence SEQ ID NO:9 and a recombinant TCRB gene encoding CDR3 sequence SEQ ID NO:4.

3. The isolated recombinant human T cells of claim 1 comprising (a) a TCRA gene encoding SEQ ID NO:10 and a TCRB gene encoding SEQ ID NO:11;
(b) a TCRA gene encoding SEQ ID NO:12 or SEQ ID NO:13 and a TCRB gene encoding SEQ ID NO:14; (c) a TCRA gene encoding SEQ ID NO:15 and a TCRB gene encoding SEQ ID NO:16; or (d) a TCRA gene encoding SEQ ID NO:17 and a TCRB gene encoding SEQ ID NO:18.

4. A plurality of isolated recombinant human T cells enriched for recombinant human T cells comprising a recombinant T cell receptor alpha (TCRA) gene encoding CDR3 sequence CAPRVTGGGNKLTF (SEQ ID NO:5), CAVRDQRDDKIIF (SEQ ID NO:6), CAVRAPSGSARQLTF (SEQ ID NO:7), CALTLSGSARQLTF (SEQ ID NO:8), or CAASAPGRTDKLIF (SEQ ID NO:9).

5-9. (canceled)

10. A population of recombinant T cells prepared by a process comprising:
identifying a human T cell clonotype that specifically binds an identified portion of HPV E6 or E7 polypeptides by a process comprising:
(a) isolating T cells from a human who has been vaccinated with an immunogen comprising an HPV E6 or E7 polypeptide or a plurality of peptides from HPV E6 or E7;
(b) stimulating the T cells with one or more peptides that collectively constitute less than all of the sequence of the polypeptide or less than all of the plurality of peptides;
(c) identifying one or more T cell clones that are stimulated by the one or more peptides; and
(d) determining a nucleotide sequence of T cell receptor (TCR) alpha or TCR beta gene or a segment of TCR alpha or TCR beta of the one or more clones to identify a clonotype having a particular TCR alpha or beta gene or a variable region segment of TCR alpha or beta gene;
producing a recombinant vector encoding TCR alpha or beta or both or a segment thereof corresponding to the clonotype; and
transfecting human T cells ex vivo with the vector to generate recombinant T cells that specifically respond to HPV E6 or E7 or a portion of HPV E6 or E7.

11. The recombinant T cells of claim 10 wherein step (c) comprises by a process comprising an interferon gamma (IFNG) enzyme-linked immunospot (ELISPOT) assay.

12. The recombinant T cells of claim 10 wherein the method further comprises amplifying the recombinant T cells ex vivo.

13. A method of treating a human for a cancer caused by human papilloma virus (HPV) or other disease caused by HPV comprising:
administering to a human afflicted with a cancer caused by HPV or other disease caused by HPV:
(A) a plurality of isolated recombinant human T cells enriched for recombinant human T cells comprising a recombinant T cell receptor beta (TCRB) gene encoding CDR3 sequence CASSPTSGGLTWDEQYF (SEQ ID NO:1) or CASSHNSGREGNEQFF (SEQ ID NO:2) or CASSFPGENEQFF (SEQ ID NO:3) or CASSWEAGQETQYF (SEQ ID NO:4); or
(B) a plurality of isolated recombinant human T cells enriched for recombinant human T cells comprising a recombinant T cell receptor alpha (TCRA) gene encoding CDR3 sequence CAPRVTGGGNKLTF (SEQ ID NO:5), CAVRDQRDDKIIF (SEQ ID NO:6), CAVRAPSGSARQLTF (SEQ ID NO:7), CALTLSGSARQLTF (SEQ ID NO:8), or CAASAPGRTDKLIF (SEQ ID NO:9); or
(C) a population of recombinant T cells prepared by a process comprising:
identifying a human T cell clonotype that specifically binds an identified portion of HPV E6 or E7 polypeptides by a process comprising:
(a) isolating T cells from a human who has been vaccinated with an immunogen comprising an HPV E6 or E7 polypeptide or a plurality of peptides from HPV E6 or E7;
(b) stimulating the T cells with one or more peptides that collectively constitute less than all of the sequence of the polypeptide or less than all of the plurality of peptides;
(c) identifying one or more T cell clones that are stimulated by the one or more peptides; and
(d) determining a nucleotide sequence of T cell receptor (TCR) alpha or TCR beta gene or a segment of TCR alpha or TCR beta of the one or more clones to identify a clonotype having a particular TCR alpha or beta gene or a variable region segment of TCR alpha or beta gene;
producing a recombinant vector encoding TCR alpha or beta or both or a segment thereof corresponding to the clonotype; and
transfecting human T cells ex vivo with the vector to generate recombinant T cells that specifically respond to HPV E6 or E7 or a portion of HPV E6 or E7.

14. The method of claim 13 wherein the administered T cells are prepared by a process comprising:
collecting T cells from the human; and
transforming the T cells with a recombinant vector ex vivo to generate recombinant T cells.

15. The method of claim 14 wherein the method further comprises amplifying the recombinant T cells ex vivo.

16. A bifunctional protein comprising:
a CD3-binding domain; and
an HPV 16 E6-91-115-binding domain;
wherein the protein comprises a single chain Fv that comprises the CD3-binding domain and CDR3 segments of TCR alpha and TCR beta polypeptides that form at least a portion of the 91-115-binding domain.

17. The bifunctional protein of claim 16 wherein the protein comprises two polypeptides, (a) and (b), connected by a cysteine disulfide, wherein polypeptide (a) is a fusion polypeptide comprising a TCR alpha segment and a single chain Fv comprising the CD3-binding domain, and polypeptide (b) comprises a TCR beta segment, wherein the TCR alpha and TCR beta segments form the E6 91-115 binding domain.

18. The bifunctional protein of claim 17 wherein the fusion protein comprises (a) a TCR alpha CDR3 sequence SEQ ID NO:5 and a TCR beta CDR3 sequence SEQ ID NO:2; (b) a TCR alpha CDR3 sequence SEQ ID NO:6 or 7 and TCR beta CDR3 sequence SEQ ID NO:1; (c) a TCR alpha CDR3 sequence SEQ ID NO: and TCR beta CDR3 sequence SEQ ID NO:3; or (d) a TCR alpha CDR3 sequence SEQ ID NO:9 and TCR beta CDR3 sequence SEQ ID NO:4.

19. The bifunctional protein of claim 17 wherein polypeptide (a) comprises SEQ ID NO:27 and polypeptide (b) comprises SEQ ID NO:26.

20. A method of treating a human for a cancer caused by HPV or other disease caused by HPV comprising:
administering the bifunctional protein of claim 16 to a human afflicted with a cancer caused by HPV or other disease caused by HPV.


SEQUENCE LISTING (27 sequences)

SEQ ID NO: 1 - protein, 17 aa, Homo sapiens
CASSPTSGGLTWDEQYF

SEQ ID NO: 2 - protein, 16 aa, Homo sapiens
CASSHNSGREGNEQFF

SEQ ID NO: 3 - protein, 13 aa, Homo sapiens
CASSFPGENEQFF

SEQ ID NO: 4 - protein, 14 aa, Homo sapiens
CASSWEAGQETQYF

SEQ ID NO: 5 - protein, 14 aa, Homo sapiens
CAPRVTGGGNKLTF

SEQ ID NO: 6 - protein, 13 aa, Homo sapiens
CAVRDQRDDKIIF

SEQ ID NO: 7 - protein, 15 aa, Homo sapiens
CAVRAPSGSARQLTF

SEQ ID NO: 8 - protein, 14 aa, Homo sapiens
CALTLSGSARQLTF

SEQ ID NO: 9 - protein, 14 aa, Homo sapiens
CAASAPGRTDKLIF

SEQ ID NO: 10 - protein, 273 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAPRVTGGGNKLTFGTGTQLKVELNIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 11 - protein, 314 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSHNSGREGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - protein, 268 aa, Homo sapiens
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRDQRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 275 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPSGSARQLTFGSGTQLTVLPDIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 315 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPTSGGLTWDEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 15 - protein, 273 aa, Homo sapiens
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALTLSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 311 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFPGENEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 273 aa, Homo sapiens
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASAPGRTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 311 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSWEAGQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - protein, 10 aa, Human papillomavirus
TIHDIILECV

SEQ ID NO: 20 - protein, 10 aa, Human papillomavirus
FAFRDLCIVY

SEQ ID NO: 21 - protein, 15 aa, Human papillomavirus
YGTTLEQQYNKPLCD

SEQ ID NO: 22 - protein, 15 aa, Human papillomavirus
EQQYNKPLCDLLIRC

SEQ ID NO: 23 - protein, 15 aa, Human papillomavirus
KPLCDLLIRCINCQK

SEQ ID NO: 24 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 25 - protein, 248 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPSGSARQLTFGSGTQLTVLPDIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQN

SEQ ID NO: 26 - protein, 279 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPTSGGLTWDEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVL

SEQ ID NO: 27 - protein, 522 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPSGSARQLTFGSGTQLTVLPDIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNQPAMAQVQLQESGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTPKLEEGEFSEARVDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTGSEQKLISEEDLNSHHHHHH